(19) United States
(12) Patent Application Publication
(10) Pub. No.: US 2004/0171853 A1
(43) Pub. Date: Sep. 2, 2004

(54) Microbial process to prepare5-androsten-3beta,7alpha, 15alpha-triol-17-one and related analogues

(76) Inventors: Michael Jon White, Portage, MI (US); Ivan Gale Gilbert, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/772,527

(22) Filed: Feb. 5, 2004

Related U.S. Application Data
(60) Provisional application No. 60/446,047, filed on Feb. 7, 2003.

(51) Int. Cl. C12P 33/06; C07J 1/00

(52) U.S. Cl. 552/615; 435/58

(57) ABSTRACT

The invention relates to a fungal process to prepare 5-androsten-3β,7α,15α-triol-17-one and related analogues.

### CROSS REFERENCE TO RELATED APPLICATION

This application claims the benefit of U.S. provisional application Serial No. 60/446,047 filed on 7 Feb. 2003, under 35 USC 119(e)(i), which are incorporated herein by reference in their entirety.

### FIELD OF THE INVENTION

The present invention relates to a fungal process to prepare 5-androsten-3β,7α,15α-triol-17-one and related analogues.

### BACKGROUND

Steroid intermediates are often useful in the production of pharmaceutical agents. 5-androsten-3β,7α,15α-triol-17-one is a steroid intermediate useful for producing certain steroid lactone analogues used as aldosterone receptor antagonists, diuretic agents, and for women's health. For example, 5-androsten-3β,7α,15α-triol-17-one can be used in the chemical synthesis of drospirenone (German Patent No. DE 5569652).

Kieslich (German Patent No. DE 2746298) and Petzoldt et al. (Angew. Chem. 95(5), 413-414, 1983; U.S. Pat. No. 4,435,327) describe microbial processes for the conversion of 5-androsten-3-ol-17-one and related analogues to 5-androsten-3β,7α,15α-triol-17-one using filamentous fungi belonging to the genus Colletotrichum. Kieslich (op. cit.) describes a method using Colletotrichum phomoides ifo 5257 and a concentration of approximately 1 g 5-androsten-3β-ol-17-one per liter of fermentation broth. Petzoldt et al. (op. cit.) using Colletotrichum lini cbs 112.21, also describe a method using 5-androsten-3-ol-17-one at approximately 1 g/l. While the yields are good, approximately 76% and 86% respectively, the amount of material converted using these methods is relatively low and probably impractical. Okada and Saito (Steroids 6(5), 651-657, 1965), and Okada et al. (Yakugaku Zasshi 85, 816, 1965) have used the filamentous fungus Gibberella saubinetti to convert 5-androsten-3β-ol-17-one to 5-androsten-3β,7α,15α-triol-17-one. Again, very low concentrations of 5-androsten-3β-ol-17-one were used, and multiple side-products were identified. Kolek (Journal of Steroid Biochemistry and Molecular Biology 71, 83-90, 1999) affected both 7α- and 15α-hydroxylation using 5-ene steroids and the filamentous fungus Fusarium culmorum. However, 7α,15α-dihydroxylation was only achieved when the steroid substrates lacked oxygen function at either the C-3 or the C-17 positions, or had a progesterone-type side chain at C-17, i.e., 5-androsten-17-one, 5-androsten-3β-ol, and pregnenolone. In all three cases, the level of 7α,15-dihydroxylation was low either being accompanied by other side-products or a high proportion of unconverted starting material. When 5-androsten-3β-ol-17-one was used, 5-androsten-3β,7α-diol-17-one was the major product. No evidence of 7α,15α-dihydroxylation was observed. Similarly, Defaye et al. (Journal of Steroid Biochemistry 9, 331-336, 1978) showed that Fusarium graminearum could affect 7α- and 15α-hydroxylations on steroids, but only one or the other. Thus, there is a need for a practical process to prepare 5-androsten-3β,7α,15α-triol-17-one and related analogues by biotransformation.

### SUMMARY OF INVENTION

The present invention provides a practical fungal method for 7α,15α-dihydroxylation of 5-androsten-3β-ol-17-one and other related analogues of the general Formula I to yield 5-androsten-3β,7α,15α-triol-17-one and other related analogues of the general Formula II.

### DETAILED DESCRIPTION OF THE INVENTION

In the detailed description, the following definitions are used.

The term “alkyl” means, unless otherwise stated, a straight or branched chain. Examples include, but are not limited to, groups such as methyl, ethyl, n-propyl, isopropyl, n-butyl, t-butyl, isobutyl, sec-butyl, n-pentyl, isopentyl, neopentyl and the like.

The term “biotransformation” means transformation of chemical compounds within a living system (Webster's New Collegiate Dictionary, Merriam-Webster pub., 1980).

Steroid compounds of Formula I are hydroxylated at the 7- and 15-position

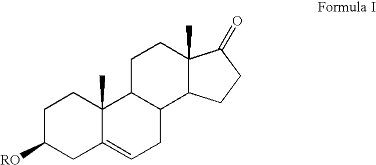

wherein R is —H or —CO—R1;

R1 is C1-C5 alkyl;

to produce steroid compounds of Formula II.

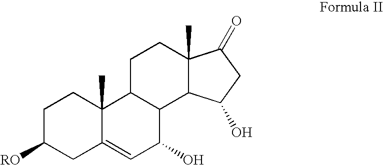

wherein R and R₁ are as defined above.

A filamentous fungus species of the genus Fusarium capable of the biotransformation of compounds of Formula I to compounds of Formula II is used in the invention process. Preferably, Fusarium culmorum is used, for example, Fusarium culmorum UC 16069. The fungus is grown in submerged culture under aerobic conditions, using art-recognized procedures, and the 7α,15α-dihydroxylation reaction performed in situ. The procedure of EXAMPLE 1, with appropriate modifications known to those skilled in the art as necessary, may be used to determine species capable of the biotransformation.

The fungus may be cultured under conditions set forth in EXAMPLE 1 using the ingredients specified, or other suitable carbon and nitrogen sources as are known to those skilled in the art. Carbon sources may be selected from, but not restricted to, the sub-groups consisting of monosaccharides, disaccharides, trisaccharides, polysaccharides, and sugar alcohols. Preferably, the monosaccharide glucose is used at a concentration from 2 g/L to 100 g/L, but typically 5 g/L to 60 g/L. Nitrogen sources may be selected from, but not restricted to, the groups consisting of nitrogen-containing organic substances such as casein, cornsteep liquor, meat extract, peptone, soy protein hydrolysate, soy flour, and yeast extract, and nitrogen-containing inorganic compounds such as nitrates and inorganic ammonium salts. Typically, the nitrogen-containing organic substance soy flour is used at a concentration from 5 g/L to 50 g/L, but typically 10 g/L to 35 g/L. A primary and secondary vegetative seed procedure is used in preparation for the fungal 7α,15α-dihydroxylation. Alternatively, a primary vegetative seed can be used directly to inoculate biotransformation media.

Primary vegetative seed cultures may be incubated for a period of 24 to 96 hours (preferably 48 to 72 hours) at a temperature between 20° C. and 37° C. (preferably 28° C.), and a pH between 3.0 and 7.5. Secondary vegetative seed medium is inoculated with 0.006% to 0.1% (v/v) primary vegetative seed culture, but typically 0.012% (v/v), and incubated for a period of 36 to 72 hours (usually 48 to 60 hours) at a temperature between 20° C. and 37° C. (preferably 28° C.). The pH of the secondary seed medium can be between 2.5 and 7.0 (preferably between 3.0 and 5.0). The biotransformation medium, which can be the same or similar to the secondary vegetative seed medium, is inoculated with 1% to 10% (v/v) secondary vegetative seed culture (preferably 3% to 5%). After an initial incubation period of 12 to 72 hours (preferably 16 to 24 hours), steroid substrates of Formula I, preferably micronized, are added to the biotransformation culture. Micronized steroid substrates of Formula I can be added as a dry powder or an aqueous slurry, either as a single addition, a series of additions, or a continual feed. It is preferred to use the micronized steroid substrates of Formula I at a concentration greater than 1 g/L, more preferably greater than 2.5 g/L, even more preferably greater than 5 g/L. Biotransformation of steroid substrates of Formula I to form steroid products of Formula II is allowed to proceed for between 2 and 10 days, but typically 3 to 6 days. The progress of the biotransformation may be followed using analytical methods known to those skilled in the art. A chromatographic method is described in EXAMPLE 1. The method may also be used to identify other species within the genus Fusarium and strains of fungi capable of performing the biotransformation.

The rate and extent of 7α,15α-dihydroxylation is improved by:

(i) culturing the selected fungus, and performing the biotransformation in the presence of a surfactant. The surfactant is selected from the group of non-ionic detergents including non-ionic amides, non-ionic esters such as ethoxylated alkyl phenols and polyethylene sorbitan esters, emulsifying waxes, non-ionic ethoxylates, tristyrylphenol ethoxylates, alcohol ethoxylates, ethoxylated mercaptans, capped ethoxylates, block copolymers, and reverse copolymers. Preferably the sub-groups consisting of ethoxylated alkylphenols and polyoxyethylenesorbitan esters are used. More preferably, octylphenoxypolyethoxyethanol or nonylphenoxypolyethoxyethanol is used at a concentration from 0.1 mL/L to 4 mL/L, but typically 0.25 mL/L to 2 mL/L;

(ii) culturing the selected fungus, and performing the biotransformation in the presence of a natural oil. The natural oil is selected from, but not restricted to, the group consisting of caster oil, corn oil, cottonseed oil, lard oil, linseed oil, olive oil, peanut oil, rapeseed oil, safflower seed oil, soybean oil, sunflower seed oil, and wheat germ oil. Preferably, soybean oil is used at a concentration from 1 mL/L to 40 mL/L, but typically 5 mL/L to 30 mL/L; and

(iii) using a combination of the methodologies identified in (i) and (ii).

Once the biotransformation of steroid substrates of Formula I to steroid products of Formula II is complete, steroid compounds of Formula II can be isolated using the solvents and conditions set forth in EXAMPLE 1, or any one of a number of art-recognized procedures. Preferably, filtered or centrifuged beer solids are extracted using a water-miscible organic solvent such as methanol or acetone at temperatures from 15° C. to 55° C., but typically 30° C. to 35° C. The preferred extraction solvent mixture is 80% acetone/20% water. The crude product of Formula II is generated by evaporative crystallization to remove the organic solvent, and cooling. The spent aqueous liquor is discarded. Crude crystalline product of Formula II is purified by carbon treatment and crystallization. It is preferred that the carbon treatment and subsequent crystallization be done using a solvent selected from, but not restricted to, the group consisting of methanol, acetone or n-butyl acetate. The preferred carbon-treatment solvent is methanol. After removal of carbon by filtration, the purified product of Formula II is recovered by solvent exchange, evaporation and cooling. The preferred crystallization solvent is n-butyl acetate.

### EXAMPLE

Without further elaboration, it is believed that one skilled in the art can, using the preceding descriptions, practice the present invention to its fullest extent. The following detailed example describes how to prepare the various compounds and/or perform the various processes of the invention and are to be construed as merely illustrative, and not limitations of the preceding disclosure in any way whatsoever. Those skilled in the art will promptly recognize appropriate variations from the procedures, both as to reactants and as to reaction conditions and techniques.

### Example 1

### Biotransformation of 5-androsten-3β-ol-17-one (Formula I, Where R=Hydrogen) to 5-androsten-3β,7α,15α-triol-17-one (Formula II, Where R=Hydrogen)

The biotransformation of 5-androsten-3β-ol-17-one to 5-androsten-3β,7α,15α-triol-17-one was performed using a submerged culture of Fusarium culmorum UC 16069 at a 10-L fermentation scale.

(A) Primary-Seed Stage

Frozen vegetative cells of Fusarium culmorum UC 16069 were thawed, transferred to potato-dextrose-agar plates (PDA), and incubated at 28° C. for 72 hours. Single mycelial-plugs (6-7 mm diameter) were used to inoculate siliconized 500-mL stippled shake flasks containing 100 mL primary-seed medium. Primary-seed medium consists of (per liter of RO water): dextrin, 50 g; soyflour, 35 g; glucose, 5 g; coboalt chloride hexahydrate, 2 mg; silicone defoamer (SAG 471), 0.5 mL; pre-sterilization pH 7.0-7.2, adjusted with sodium hydroxide (2N). Primary-seed medium was sterilized for 30 minutes at 121° C. using an autoclave. Fusarium culmorum UC 16069 is incubated for 48 hours at 28° C., using a controlled-environment incubator-shaker set at 270 rpm. (1″ orbital stroke).

(B) Secondary-Seed Stage

One hundred milliliter secondary-seed fermentations in siliconized 500-mL stippled shake flasks were inoculated using vegetative primary-seed culture (approximately 0.012% [v/v] inoculation rate). Secondary-seed medium contains (per liter of RO water): glucose, 60 g; soyflour, 25 g; soybean oil, 30 mL; magnesium heptahydrate, 1 g; potassium dihydrogen phosphate, 0.74 g; octylphenoxypolyethoxyethanol, 0.25 mL; silicone defoamer (SAG 471), 0.5 mL; pre-sterilization pH 3.95-4.00, adjusted with concentrated sulfuric acid. Secondary-seed medium was sterilized for 30 minutes at 121° C. using an autoclave. Fusarium culmorum UC 16069 was incubated at 28° C. for 48-50 hours, using a controlled-environment incubator-shaker set at 270 rpm. (1″ orbital stroke).

(C) Steroid Biotransformation

Ten-liter steroid-biotransformation fermentations were inoculated using 500 mL vegetative secondary-seed culture (5% [v/v] inoculation rate). Steroid-biotransformation medium was essentially the same as secondary-seed medium, with the exception that the octylphenoxypolyethoxyethanol was increased from 0.25 mL/L to 2 mL/L. The fermentors, containing steroid-biotransformation medium, were sterilized for 20 minutes at 121° C. using both jacket and injection steam. The agitation rate during sterilization was 200 r.p.m. Post-sterilization, the medium pH was adjusted to 4.0 using sterile sulfuric acid (5%). Fusarium culmorum UC 16069 was incubated at 28° C. using the following initial parameters: agitation, 200 r.p.m.; back pressure=5 psig; airflow=2.5 SLM (0.25 VVM); low dissolved oxygen set-point, 50%; pH control, none. When the dissolved oxygen first dropped to 50%, the airflow was increased to 5 SLM (0.5 VVM). When the culture reached low dissolved oxygen again, 50% dissolved oxygen was maintained using agitation control. At 16 to 17 hours post-inoculation, 200 g micronized 5-androsten-3β-ol-17-one, slurried in a minimal volume of 0.2% octylphenoxypolyethoxyethanol, was added to the 110-L fermentation.

Biotransformation cultures were assayed on a daily basis for 5-androsten-3β,7α,15α-triol-17-one using thin layer chromatography. One milliliter of whole beer was extracted with 19 mL methanol. Cells were separated from the aqueous-methanol mixture by centrifugation (3,000×g for 10 minutes), and 5μ microliters applied to a thin layer chromatography plate. The thin layer chromatography plate was developed in cyclohexane:ethyl acetate:methanol (90:60:15) and the product visualized by spraying the thin layer chromatography plate with 50% sulfuric acid, followed by charring in an oven. Product was compared with authentic standard, which turns blue on spraying with 50% sulfuric acid. Biotransformation of 5-androsten-3β-ol-17-one to 5-androsten-3β,7α,15α-triol-17-one was complete approximately 6 days post-inoculation.

(D) Isolation Procedure

The whole beer solids from two fermentations were recovered by centrifugation. The liquid was discarded. The rich solids were extracted with 20 liters of 80% acetone/20% water at 30° C. to 35° C. and then re-extracted with 10 liters of 80% acetone/20% water at 30° C. to 35° C. The extracts were pooled, filtered, and concentrated by distillation to remove acetone generating an aqueous slurry of crude crystals. The crude crystals were recovered by filtration and the mother liquor was discarded. The water-wet crystals were dissolved in 3 liters of methanol and then decolorized with 25 grams of Darco G-60 carbon for 1 hour. After filtration to remove carbon, the filtrate was concentrated by evaporation to crystallize the product. The methanol was removed further by adding 500 mL of n-butyl acetate and concentrating to a thick crystal slurry. The crystals were filtered, washed with n-butyl acetate, and dried to give 241.6 grams of crude crystalline 5-androsten-3β,7α,15α-triol-17-one.

The crude crystals were dissolved in methanol. An equal volume of n-butyl acetate was added and the mixture was concentrated by evaporation to remove methanol generating a thick slurry. The crystals were filtered, washed with n-butyl acetate, and dried to give 190.5 grams of purified crystalline 5-androsten-3β,7α,15α-triol-17-one.

We claim:

1. A process for the production of steroid compounds of Formula II 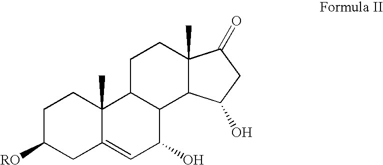
wherein R is:
—H or —CO—R1;
wherein R1 is C1-C5 alkyl;
by fungal dihydroxylation of steroid compounds of Formula I 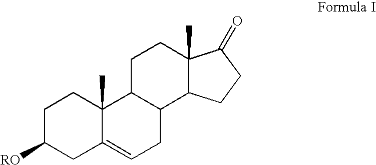
comprising contacting a steroid of Formula I with a biotransformation culture containing a species of the genus Fusarium capable of 7α,15α-dihydroxylation.

2. A process for producing steroids of Formula II according to claim 1 wherein the 7α,15α-dihydroxylation is achieved with the strain Fusarium culmorum UC 16069.

3. A process for producing steroids of Formula II according to claim 2 further comprising the steps of:
a) preparing a primary seed culture of Fusarium;
b) preparing a secondary seed culture using the culture of step a);
c) preparing a tertiary culture for biotransformation using the culture of step b);
d) adding a steroid of Formula I to the culture of step c);
e) collecting the solids from the culture of step d); and
f) extracting the solids of step d);

4. A process for producing steroids of Formula II according to claim 1 wherein the biotransformation culture contains a surfactant.

5. A process for producing steroids of Formula II according to claim 4 wherein the surfactant is a non-ionic detergent selected from the group consisting of non-ionic amides, non-ionic esters such as ethoxylated alkyl phenols, polyethylene sorbitan esters, emulsifying waxes, non-ionic ethoxylates, tristyrylphenol ethoxylates, alcohol ethoxylates, ethoxylated mercaptans, capped ethoxylates, block copolymers, and reverse copolymers.

6. A process for producing steroids of Formula II according to claim 5 wherein the surfactant is an ethoxylated alkyl phenol.

7. A process for producing steroids of Formula II according to claim 6 wherein the ethoxylated alkyl phenol is octylphenoxypolyethoxyethanol.

8. A process for producing steroids of Formula II according to claim 6 wherein the ethoxylated alkyl phenol is nonylphenoxypolyethoxyethanol.

9. A process for producing steroids of Formula II according to claim 1 wherein the biotransformation culture contains a natural oil.

10. A process for producing steroids of Formula II according to claim 9 wherein the natural oil is selected from the group consisting of caster oil, corn oil, cottonseed oil, lard oil, linseed oil, olive oil, peanut oil, rapeseed oil, safflower seed oil, soybean oil, sunflower seed oil, and wheat germ oil.

11. A process for producing steroids of Formula II according to claim 10 wherein the natural oil is soybean oil.
